(19) United States
(12) Patent Application Publication
(10) Pub. No.: US 2015/0238143 A1
(43) Pub. Date: Aug. 27, 2015

(54) Helmet Head Impact Tracking and Monitoring System

(71) Applicant: Russell Meurer, Las Vegas, NV (US)

(72) Inventor: Russell Meurer, Las Vegas, NV (US)

(21) Appl. No.: 14/665,812

(22) Filed: Mar. 23, 2015

Related U.S. Application Data
(63) Continuation of application No. 14/632,080, filed on Feb. 26, 2015, now abandoned.
(60) Provisional application No. 61/945,455, filed on Feb. 27, 2014.

(51) Int. Cl. A61B 5/00; A61B 19/00; A61B 5/11; A42B 3/04

(52) U.S. Cl. CPC A61B 5/6803; A42B 3/046; A42B 3/0453; A61B 19/46; A61B 5/7264; A61B 5/7282; A61B 5/4064; A61B 5/11; A61B 5/742; A61B 19/44; A61B 5/0004; A61B 5/0022; A61B 2019/465; A61B 2562/0219; A61B 2562/227; A61B 2560/0475; A61B 2560/0487

(57) ABSTRACT

A headgear-based system for tracking and monitoring the occurrence of impacts to a user's head. The present system includes a sensor array disposed on the interior surface of a helmet or other headgear that is adapted to detect when the head contained within the helmet has sustained an impact above a pre-determined threshold value, indicating the potential for the user to have suffered a concussion. When a qualifying impact has been detected, the system uploads the data pertaining to the impact to a computer system for access by the user or a third party and activates an indicator disposed on the helmet to notify surrounding individuals that the user may have suffered from a concussion and requires further evaluation. In one embodiment, the indicator is a light disposed on the exterior surface of the helmet or other headgear.

### CROSS REFERENCE TO RELATED APPLICATION

This application is a continuation of U.S. Nonprovisional patent application Ser. No. 14/632,080, which claims priority to Provisional Application No. 61/945,455 filed on Feb. 27, 2014. The above identified patent applications are herein incorporated by reference in its entirety to provide continuity of disclosure.

### FIELD OF THE INVENTION

The present invention relates to head trauma monitoring systems. More specifically, the present invention relates to headgear comprising sensors to detect when user has suffered an impact to his or her head for the purpose of monitoring the occurrence of head trauma.

### BACKGROUND OF THE INVENTION

As our medical understanding of concussions has grown, society has become more and more conscious of the dangers caused by repeated, forceful impacts to the head and the need to track and monitor such impacts over an extended period of time. Although our knowledge is still imperfect, research indicates that individuals who suffer a concussion need to be immediately removed from the activity and monitored because the brain is even more susceptible to damage in the time period immediately following the concussion.

However, it can be difficult to determine when an individual has suffered a concussion because individuals manifest the symptoms of concussions in different ways. The symptoms of a concussion can range from obvious, e.g. passing out, to subtle, e.g. slight balance impairment, and can last between hours and months. There is little consistency from person-to-person as to how the symptoms manifest; therefore, a concussion can be difficult to diagnose without further evaluation, monitoring, and testing. The analysis of whether an individual has suffered a concussion is particularly difficult in a sports context where coaches and medical personnel have to quickly and accurately assess players' mental states in a distraction-rich environment and where the players themselves may be intentionally or unintentionally attempting to hide the symptoms in order to prevent an accurate diagnosis. Furthermore, individuals need to have access to the history of the head impacts that they have suffered playing sports and performing other activities so that they can educate themselves and make intelligent, fully-informed medical decisions about their future. Therefore, there is a need in the prior art for a system that is adapted to track and monitor the occurrence of head impacts suffered when playing sports or performing other activities and adapted to provide cues or alerts to surrounding people that an individual potentially may have suffered from a concussion and needs to be evaluated.

### SUMMARY OF THE INVENTION

In view of the foregoing disadvantages inherent in the known types of head impact monitoring systems now present in the prior art, the present invention provides a head impact tracking and monitoring system wherein the same can be utilized for providing convenience for the user when playing football, riding a motorcycle, and performing other activities where the user wears a helmet and that inherently comprise a risk for the user to suffer an impacted-related head injury. The present system comprises a plurality of sensors disposed on the interior surface of a helmet or other headgear that are adapted to detect when the head contained within the helmet has sustained an impact above a pre-determined threshold value, indicating the potential for the user to have suffered a concussion. When a qualifying impact has been detected, the system uploads the data pertaining to the impact for access by the user or a third party and activates an indicator for notifying surrounding individuals that the user may have suffered from a concussion and needs to be evaluated. In one embodiment, the indicator is a light disposed on the exterior surface of the helmet or other headgear.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1A shows a cutaway view of the front of a football helmet embodiment of the present invention.

FIG. 1B shows a cutaway view of the back of a football helmet embodiment of the present invention.

FIG. 1C shows a cutaway view of the side of a football helmet embodiment of the present invention.

FIG. 2 shows a cutaway perspective view of a football helmet embodiment of the present invention.

FIG. 3 shows a cutaway perspective view of a motorcycle helmet embodiment of the present invention.

FIG. 4 shows a block diagram of the representative electronic components of the present invention.

FIG. 5 shows a flowchart of the logic of the present invention.

FIG. 6 shows a schematic diagram of the function of the present invention.

### DETAILED DESCRIPTION OF THE INVENTION

Reference is made herein to the attached drawings. Like reference numerals are used throughout the drawings to depict like or similar elements of the helmet head impact tracking and monitoring system. For the purposes of presenting a brief and clear description of the present invention, the preferred embodiment will be discussed as a football helmet. The figures are intended for representative purposes only and should not be considered to be limiting in any respect.

As used herein, “logic” refers to (i) logic implemented as computer instructions and/or data within one or more computer processes and/or (ii) logic implemented in electronic circuitry. As used herein, “computer readable medium” excludes any transitory signals, but includes any non-transitory data storage circuitry, e.g., buffers, cache, and queues, within transceivers of transitory signals.

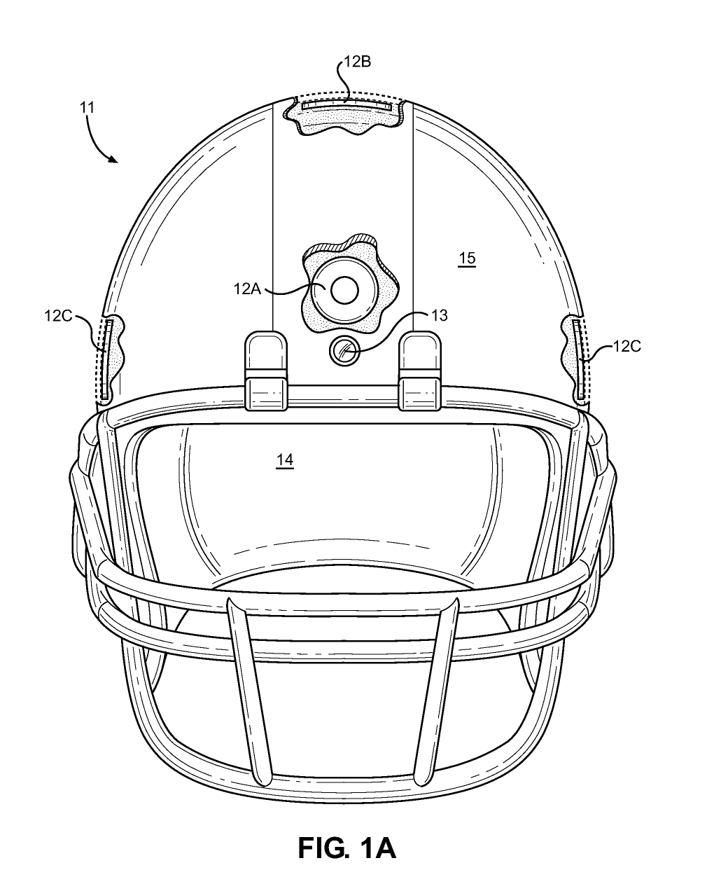

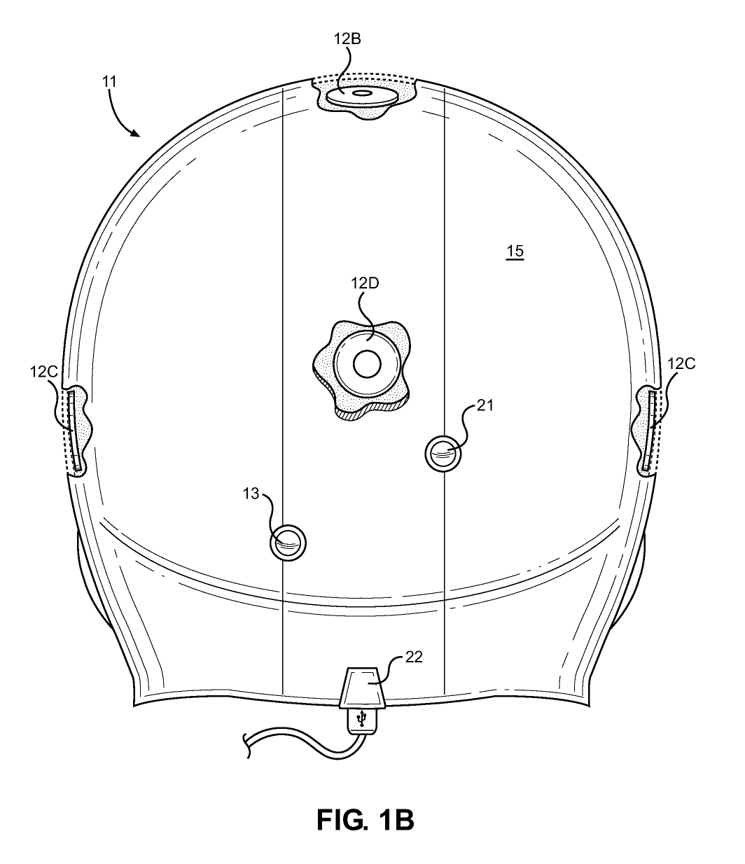

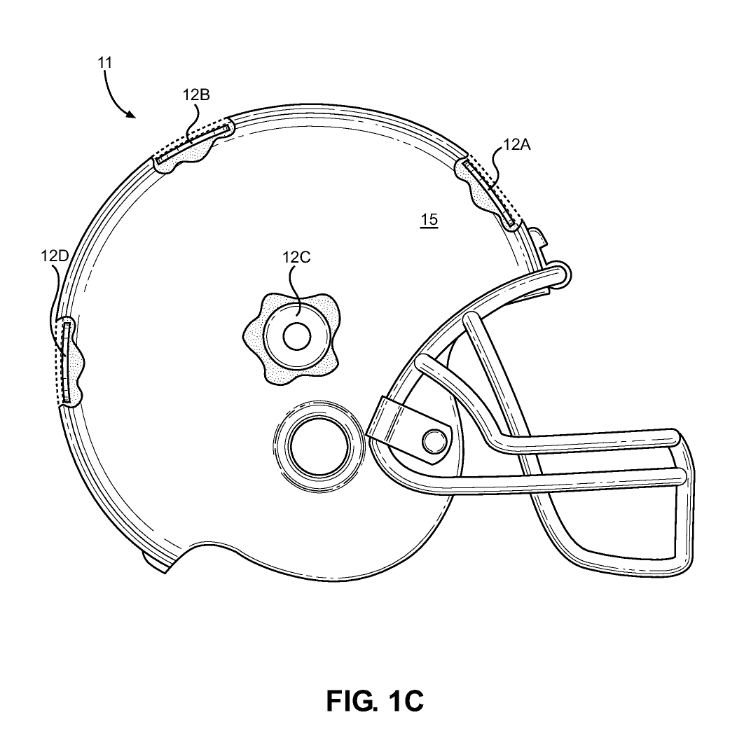

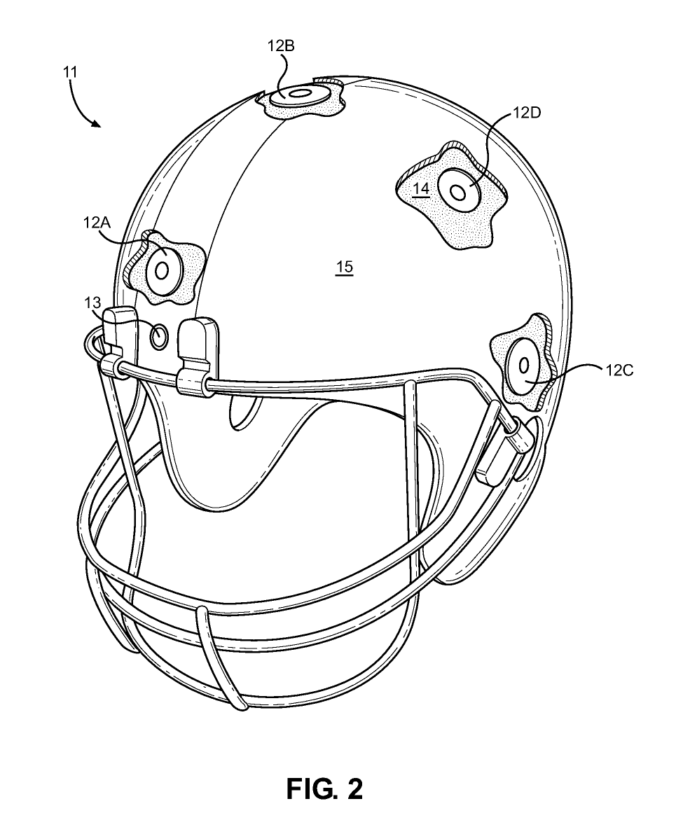

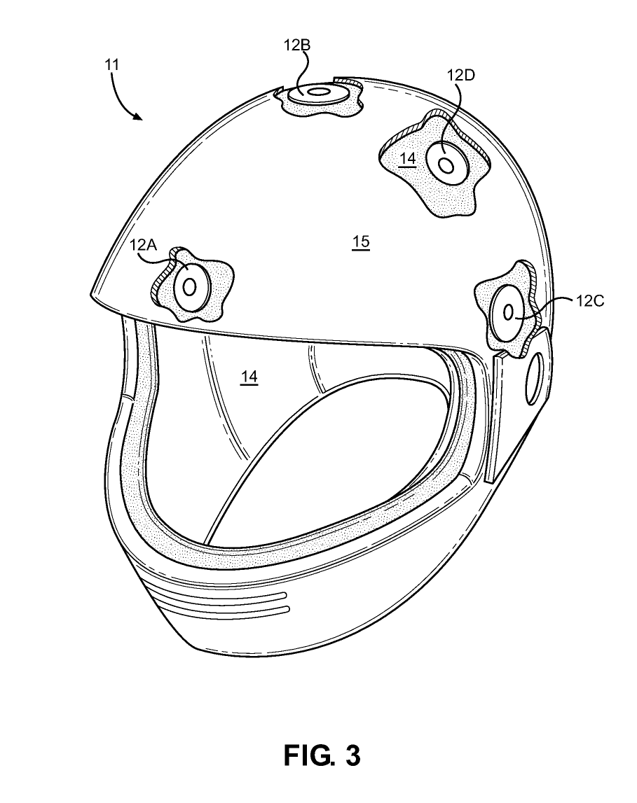

Referring now to FIGS. 1A-3, there are shown various cutaway views of alternative embodiments of the present invention. The present invention comprises a helmet 11, or another such head-worn article, and one or more impact sensors 12A, 12B, 12C, 12D disposed on the interior surface 14 of the helmet 11. In exemplary embodiments of the present invention, the helmet 11 comprises a football helmet, as shown in FIG. 1A, or a motorcycle helmet, as shown in FIG. 1B. The helmet 11 comprises additional components conventionally associated with the different types of helmets. For example, for embodiments of the present invention wherein the helmet 11 comprises a football helmet, the helmet 11 further comprises internal padding adapted to fit snugly against the head of the wearer, a face grill, and other such components. However, no claim is made as to the precise type or configuration of the helmet 11, except in that it is a head-worn article.

The impact sensors 12A, 12B, 12C, 12D comprise accelerometers or other sensors adapted to detect the magnitude of a force or pressure resulting from an impact to the helmet 11. Furthermore, the sensors 12A, 12B, 12C, 12D are arranged so that they can detect the varying levels of force imparted upon different regions of the user's head from a single impact. Because a concussion is the result of the compression of the brain against the interior of the skull and this compression is greatest at areas that are subjected to higher magnitude forces, the present invention can indicate the areas of the user's brain at which a concussion is most likely to have occurred, thereby assisting medical personnel in diagnosing and treating a concussion. In the depicted embodiment of the present invention, the sensors 12A, 12B, 12C, 12D comprise a first sensor 12A positioned on the interior surface 14 of the helmet 11 corresponding to the location of the frontal lobe of the user, a second sensor 12B corresponding to the location of the parietal lobe of the user, a third sensor 12C corresponding to the location of the temporal lobe of the user, and a fourth sensor 12D corresponding to the location of the occipital lobe of the user. However, this is merely an exemplary embodiment of the present invention and no claim is made as to the precise number or arrangement of sensors 12A, 12B, 12C, 12D within the helmet 11.

In one embodiment of the present invention, the sensors 12A, 12B, 12C, 12D are integral to the helmet 11. In this embodiment of the present invention, the sensors 12A, 12B, 12C, 12D are in electrical communication with an integral computer readable medium or memory, a processor, a wireless transceiver, and the other electronic components of the present invention via wiring disposed throughout the helmet 11. In another embodiment of the present invention, the sensors 12A, 12B, 12C, 12D are removably affixable to the interior of the helmet 11 via any removable attachment mechanism known in the prior art, such as hook-and-loop fastening material or adhesive. In this embodiment of the present invention, each of the sensors 12A, 12B, 12C, 12D comprises a wireless transceiver adapted to place the sensors 12A, 12B, 12C, 12D in wireless communication with a computer readable medium or memory, a processor, a wireless transceiver, and the other electronic components of the present invention, which may in turn be disposed within a housing that is either integral to the helmet 11 or removably affixed to the helmet 11.

The present invention further comprises an indicator 13 disposed on the helmet 11. The indicator 13 activates when the magnitude of the force of an impact exceeds a pre-programmed threshold. The indicator 13 provides individuals surrounding the wearer of the present invention with an audio or visual cue as to the fact that the wearer has been subjected to an impact that has put the wearer at risk of suffering from a concussion. This audio or visual cue makes it easier for surrounding individuals to assess whether the wearer needs to be evaluated and monitored for concussion systems. In the depicted embodiment of the present invention, the indicator 13 comprises a LED light in electrical communication with the processor; however, no claim is made as to the precise type of indicator 13. In an alternative embodiment of the present invention, the indicator 13 comprises an audio device that emits an audible alert when activated.

An alternative embodiment further comprises a connector 22 disposed on the exterior of the helmet 11. The connector 22 comprises any bus or other such device that allows for the transfer of data, such as a USB connector. The connector 22 allows data stored on the computer readable medium to be downloaded therefrom. The connector 22 provides an alternative to users to allow them to directly download the data associated with the head impacts recorded by the present invention to the wirelessly uploaded impact data. Furthermore, the connector 22 can be used to recharge the power source of the present invention.

A further alternative embodiment of the present invention comprises a safety threshold indicator 21 disposed on the helmet 11. When activated, the safety threshold indicator 21 indicates that the present invention is functioning properly and is not generating a series of false positives or false negatives when measuring impact data.

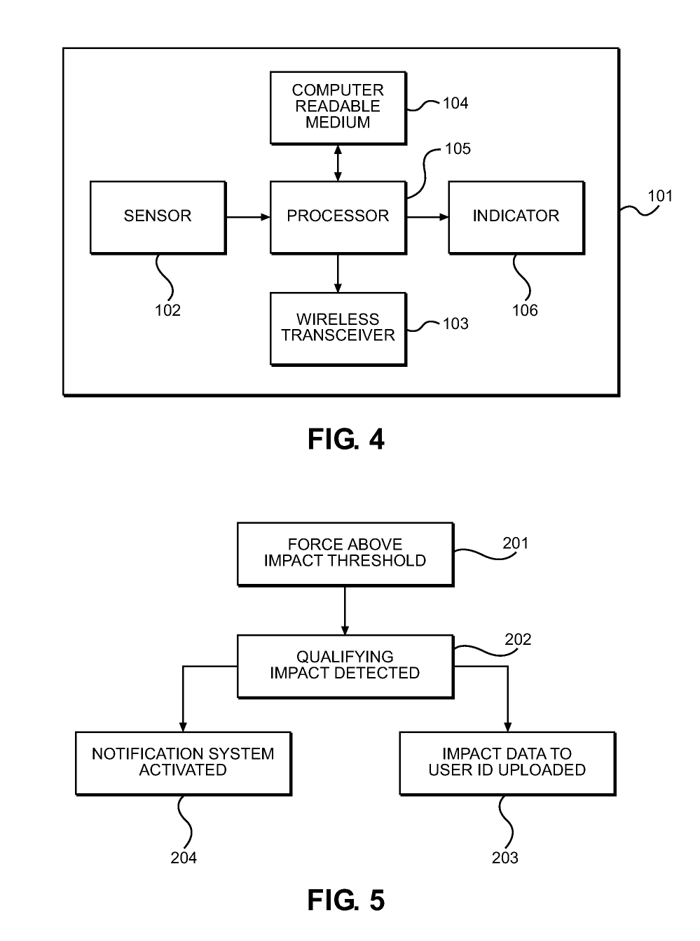

Referring now to FIG. 4, there is shown a block diagram of the representative electronic components of the present invention. The present invention comprises at least one sensor 102 disposed within the interior of the helmet, at least one wireless transceiver 103, a processor 105, a computer readable medium 104 or memory operatively connected to the processor 105 such that the logic stored within the computer readable medium 104 is adapted to be executed by the processor 105, and at least one indicator 106. The various components of the present invention are in wired, i.e. electrical, or wireless communication in different embodiments of the present invention. In one embodiment of the present invention, the sensors 102 are disposed throughout the interior surface of the helmet, the indicator 106 is disposed on the exterior of the helmet, and the computer readable medium 104, processor 105, and wireless transceiver 103 are contained within a housing that is either integral to the helmet or removably attached to the helmet. The wireless transceiver 103 comprises a direct wireless connection device, e.g. a Bluetooth connection, or a device that connects to a wireless communication network, e.g. a WiFi connection or a mobile cellular network.

The logic stored within the computer readable medium 104 is adapted to execute a series of actions or instructions based upon data received from the sensors 102. When the sensors 102 detect a force, that information is relayed to the processor 105, which then executes the logic to analyze the data. The logic is pre-programmed with a tolerance threshold, which is used to determine whether the force from the impact detected by the sensors 102 puts the wearer at risk of suffering from a concussion. If the magnitude of the force detected by the sensors 102 is above the pre-programmed threshold, then the logic causes the processor 105 to activate the wireless transceiver 103, which uploads the data to a cloud-based storage medium, a remote server, or another such type of remotely-accessible storage medium, and the indicator 106, which provides an audio or visual cue to surrounding individuals as to the fact that a forceful impact putting the wearer at risk for a concussion has occurred. In an alternative embodiment of the present invention, the logic further causes the computer readable medium 104 to store the data associated with the impact for direct retrieval from the helmet.

The present invention further comprises at least one power source in electrical communication with the various electronic components of the present invention. In one embodiment of the present invention wherein all of the electronic components are integral to the helmet, the present invention comprises a single power source in electrical communication with all of the electronic components, providing power thereto. In another embodiment of the present invention wherein the sensors 102 are removably affixed to the interior of the helmet, the indicator 106 is removably affixed to the exterior of the helmet, and the computer readable medium 104, processor 105, and wireless transceiver 103 are contained within a housing that is removably affixable to the helmet, each of these separate components comprises an independent power source. The power source of the present invention comprises a removable battery, a battery that is rechargeable via the connector, solar cells, or any other such power source known in the prior art.

Referring now to FIG. 5, there is shown a flowchart of the logic of the present invention. If the magnitude of the force from an impact is below the threshold, then the impact is not recorded. If the magnitude of the force from an impact is above the threshold 201, then the logic determines that a qualifying impact has been detected 202 and sends an activation signal to the wireless transceiver and the indicators disposed on the helmet. When the notification system is activated 204, the indicator directly attached to the helmet issues a visual or audible cue to alert individuals of the vicinity as to the fact that the wearer of the present invention has been subjected to a qualifying impact. When the wireless transceiver receives the activation signal, it uploads 203 the data associated with the impact and the user ID of the wearer to a cloud-based storage medium or other such type of remotely-accessible data storage medium via a wireless communication network or another such means of wirelessly transmitting data. In an alternative embodiment of the present invention, in addition to activating the notification system 204 and uploading the impact data and user ID 203 when an impacted is detected 202, the logic further causes the impact data and user ID to be stored on the computer readable medium so that it can be directly retrieved therefrom at a later time.

The threshold utilized by the logic to determine whether an impact has occurred may be directly input by a user or may be automatically determined via a iterative learning algorithm that determines the average magnitude of force imparted by an impact and then determines that a qualifying impact 202 has occurred when the magnitude of the force detected by the sensors exceeds this learned average magnitude. For embodiments of the present invention wherein the threshold is input by the user, this input threshold value can be determined based upon recommendations from medical personnel or studies on the effects of traumatic impacts on the brain. In a further alternative embodiment of the present invention, the threshold value can be individually set for each of the sensors. This allows for embodiments of the present invention wherein the position of the sensors corresponds to the lobes of the wearer's brain to set individualized thresholds to adjust for the potential difference in susceptibility of different lobes of the brain to injury from impacts.

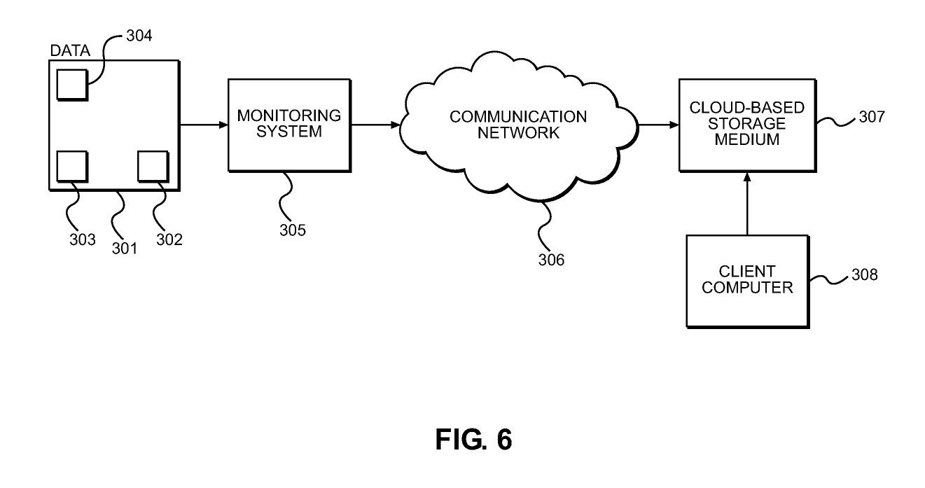

Referring now to FIG. 6, there is shown a schematic diagram of the function of the present invention. The data 301, which is uploaded by the present system 305 via a communication network 306 to a cloud-based storage medium 307 or other such type of remotely-accessible data storage medium, comprises at least the user ID 304 associated with the user, the magnitude of the detected force 302, and the sensor 303 associated with that force. This data 301 is stored on a cloud-based storage medium 307 so that it can be accessed by the user via a client computer 308 in connection therewith. The client computer 308 comprises a mobile electronic device, e.g. a cell phone or a tablet, accessing the cloud-based storage medium 307 via an app, a computer accessing the cloud-based storage medium 307 via a website, or any other such mechanism known in the prior art.

When the data 301 is uploaded to the cloud-based storage medium 307, the cloud-based storage medium 307 queries its records to determine whether data associated with the user ID 304 has been uploaded previously. If it has, then the cloud-based storage medium 307 is adapted to associate the uploaded data 301 with the previously recorded data. Because the user ID 304 corresponding to the wearer is uploaded and associated with the measured data associated with the impact 302, 303, a user can access a comprehensive history of potential concussion incidents, including at least the forcefulness of the impact to which the user was subjected and the locations of the forces imparted by the impact. Users can then provide this information to medical personnel in order to assist in diagnosis and treatment. In an alternative embodiment of the present invention, users can provide medical personnel and other such individuals with direct access to the cloud-based storage medium 307 for accessing their head impact history as recorded by the present invention. Furthermore, because the head impact data 302, 303 is uploaded with a corresponding user ID 304, this ensures that the head impact data 302, 303 is associated with the correct individual, regardless of whether that individual is using the same helmet across all of the head impact events. Users can log in or otherwise associated their user ID 304 with different helmets via any means known in the prior art.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. An impact detection system, comprising:
a helmet comprising an impact sensor adapted to measure a force of an impact on a head of a user and an indicator;
one or more processors;
a non-transitory computer readable medium operatively connected to the one or more processors;
a logic stored in said non-transitory computer readable medium that, when executed by said one or more processors, causes the impact detection system to monitor for the occurrence of head trauma by at least:
determining if the impact exceeds a threshold value;
if the impact exceeds the threshold value, then uploading data corresponding to the impact and activating the indicator.

2. The impact detection system of claim 1, wherein the impact sensor comprises a first sensor corresponding to a location of a frontal lobe of the user, a second sensor corresponding to a location of a parietal lobe of the user, a third sensor corresponding to a location of a temporal lobe of the user, and a fourth sensor corresponding to a location of an occipital lobe of the user.

3. The impact detection system of claim 1, wherein the helmet comprises a football helmet.

4. The impact detection system of claim 1, wherein the helmet comprises a motorcycle helmet.

5. The impact detection system of claim 1, wherein the impact sensor comprises an accelerometer.

6. The impact detection system of claim 1, wherein the notification system comprises a light disposed on an exterior surface of the helmet.

7. The impact detection system of claim 1, further comprising a connector disposed on the helmet to which an electronic device can be connected to directly download the data corresponding to the impact therefrom.

8. The impact detection system of claim 1, wherein the threshold value is programmable by the user.

9. The impact detection system of claim 1, wherein the data corresponding to the impact is uploaded with a user ID corresponding to the user.

10. The impact detection system of claim 1, wherein the impact sensor comprises a plurality of sensors arranged at one or more locations adapted to detect the magnitude of force from the impact at the one or more locations.

11. A method for monitoring for the occurrence of head trauma, comprising the steps of:
determining if an impact exceeds a threshold value via a helmet comprising an impact sensor adapted to measure a force of an impact on a head of a user and an indicator;
if the impact exceeds the threshold value, then uploading data corresponding to the impact and activating the indicator.

12. The impact detection system of claim 1, wherein the impact sensor comprises a first sensor corresponding to a location of a frontal lobe of the user, a second sensor corresponding to a location of a parietal lobe of the user, a third sensor corresponding to a location of a temporal lobe of the user, and a fourth sensor corresponding to a location of an occipital lobe of the user.

13. The impact detection system of claim 11, wherein the helmet comprises a football helmet.

14. The impact detection system of claim 11, wherein the helmet comprises a motorcycle helmet.

15. The impact detection system of claim 11, wherein the impact sensor comprises an accelerometer.

16. The impact detection system of claim 11, wherein the notification system comprises a light disposed on an exterior surface of the helmet.

17. The impact detection system of claim 11, further comprising a connector disposed on the helmet to which an electronic device can be connected to directly download the data corresponding to the impact therefrom.

18. The impact detection system of claim 11, wherein the threshold value is programmable by the user.

19. The impact detection system of claim 11, wherein the data corresponding to the impact is uploaded with a user ID corresponding to the user.

20. The impact detection system of claim 11, wherein the impact sensor comprises a plurality of sensors arranged at one or more locations adapted to detect the magnitude of force from the impact at the one or more locations.
